(19) United States
(12) Patent Application Publication
(10) Pub. No.: US 2024/0190833 A1
(43) Pub. Date: Jun. 13, 2024

(54) FRACTIONATION OF CANNABIS EXTRACTS

(71) Applicant: CRESCO LABS, LLC, Chicago, IL (US)

(72) Inventors: Chris Tonge, Chicago, IL (US); Tyler Finnen, Chicago, IL (US); Tamer Mohamed, Chicago, IL (US)

(21) Appl. No.: 18/078,216

(22) Filed: Dec. 9, 2022

(51) Int. Cl. C07D 311/80; B01D 21/00; B01D 21/26

(52) U.S. Cl. CPC C07D 311/80; B01D 21/0012; B01D 21/262

(57) ABSTRACT

A method of fractionation of cannabis extracts via centrifugal post extraction processing is disclosed. The method may include extracting un-activated cannabis biomass, precipitating a cannabinoid solids into a raw cannabinoid extrat, and centrifugally fractionating the bulk extract. Centrifugally fractionating the bulk extract may include placing the extract in a batch, perforated basket centrifuge, separating cannabinoid solids from liquid terpenoid/plant fat fraction; and reformulating the resulting fractions to specified cannabinoid/terpenoid contents.

### BACKGROUND

Breaking down plants such as cannabis or similar plants into useful chemicals in the plants has long been a goal. Previous approaches have used harsh chemicals and labor-intensive processes to try to break down the plants and obtain the desired chemicals. The use of chemicals and intensive processing have created a desire to create a more effective and efficient way to fractionate plant extracts into their component parts for downstream product formulation.

### SUMMARY

A method of fractionation of cannabis extracts via centrifugal post extraction processing is disclosed. The method may include extracting unactivated cannabis biomass, precipitating a mixture of acidic cannabinoid solids and terpene rich oil, and centrifugally fractionating the bulk extract into those component parts. Centrifugally fractionating the bulk extract may include placing the extract in a batch, perforated basket centrifuge, separating cannabinoid solids from liquid terpenoid/plant fat fraction; and reformulating to specified cannabinoid/terpenoid contents to yield a formulated extract product. Additional embodiments are disclosed and explained.

### BRIEF DESCRIPTION OF THE DRAWINGS

FIG. 1 may be a flow chart of a method in accordance with the claims;

FIG. 2 may be an illustration of a centrifuge;

FIG. 3 may be an illustration of centrifuge basket and center spindle;

FIG. 4 may be an illustration of centrifuge basket, backing screen and center spindle;

FIG. 5 may be an illustration of a centrifuge and filter bag;

FIG. 6 may be an illustration of a centrifuge and drain hole and drain spout;

FIG. 7 may be an illustration of a centrifuge, filter bag and badder;

FIG. 8 may be an illustration of a filter bag filled with badder;

FIG. 9 may be an illustration of a centrifuge and drain hole and drain spout;

FIG. 10 may be an illustration of a filter bag filled with partially fractionated badder;

FIG. 11 may be an illustration of a filter bag filled with completely fractionated badder;

FIG. 12 may be an illustration of a filter bag filled with completely fractionated badder in the form of cake;

FIG. 13 may be an illustration of a filter bag filled with completely fractionated badder in the form of shaken cake;

FIG. 14 may be an illustration of high terpene fluid after it is filtered by the filter bag;

FIG. 15 may be an illustration of high terpene in a container;

FIG. 16 may be an illustration of cleaning the centrifuge and basket;

FIG. 17 may be an illustration of cleaning the centrifuge, basket and drain port; and

FIG. 18 may be an illustration of cleaning the centrifuge and the lid.

### SPECIFICATION

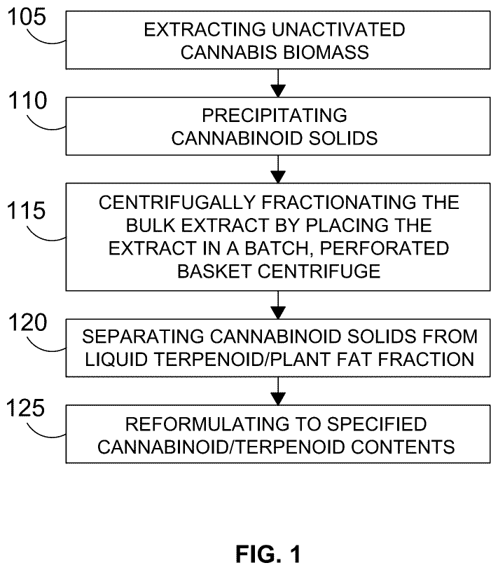

Referring now to FIG. 1, a method of fractionation of plant extracts, such as cannabis extracts, via centrifugal post extraction processing is disclosed. Technically, it is difficult to efficiently fractionalize cannabis and similar plants. In the present system, the high-level technical approach is to take advantage of the natural phase separation of unactivated cannabis extracts (into a high-terpene oil component and a high acidic cannabinoid fraction) and accelerate this separation via application of centripetal force. Naturally, extracts will slowly separate under 1 G of force. When centrifugally fractionating, the extract is spun at a high speed in a perforated basket. When extract is exposed to hundreds of Gs, it accelerates this sedimentation process. By doing this in a perforated basket, the system may be able to rapidly and efficiently separate the liquid/oil fraction from the solid acidic cannabinoid sediment.

### Startup

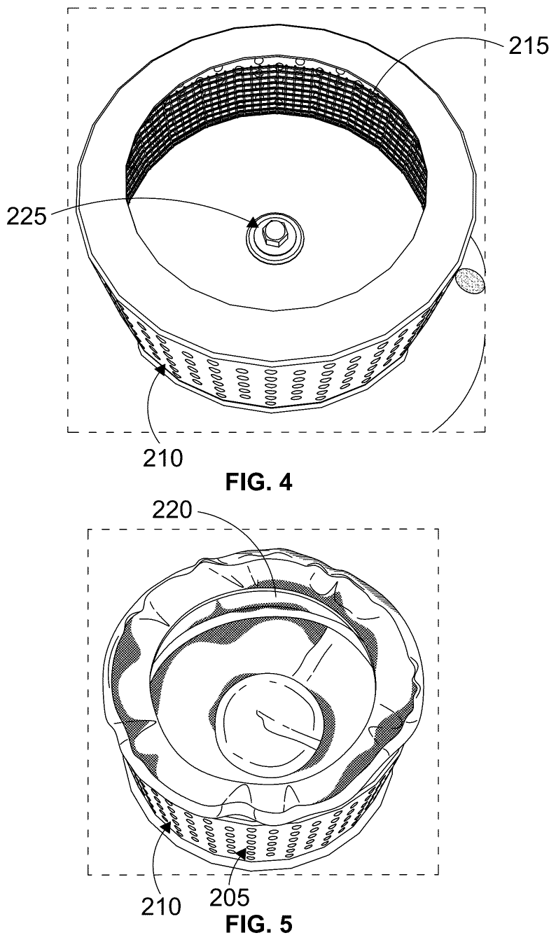

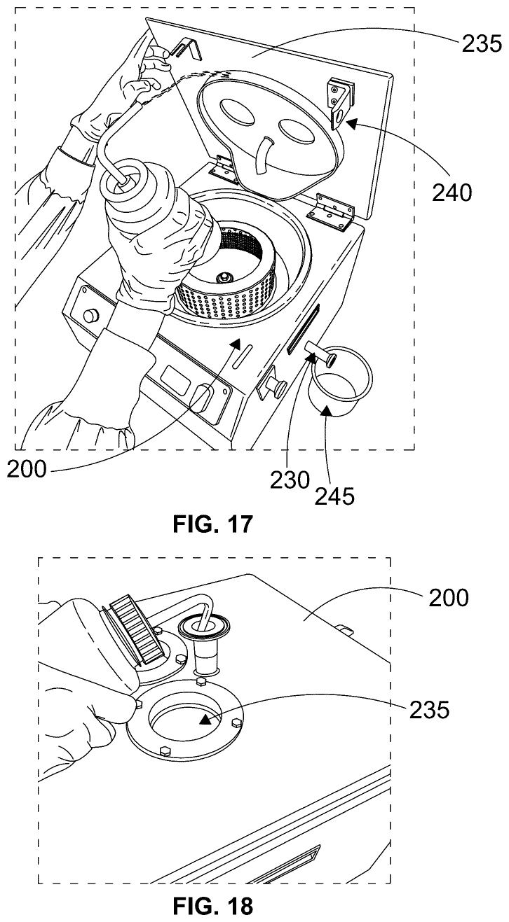

In application, a centrifuge 200 may be used to assist in the fractionation. A centrifuge 200 may turn a centrifuge basket 205 at a pre-determined or set speed for a pre-determined or set period of time. The centrifuge 200 may be turned on (using the power switch or knob). In some embodiments, power may be required to be able to access the interior of the system. The centrifuge basket 205 may also may have a perforated outside wall 210, a separate backing screen to improve filtration efficiency 215 (FIG. 3) and a filter bag 220 (FIG. 4). The centrifuge 200 also may have a center spindle 225 (FIG. 3) and a drain port 230. The centrifuge basket 205 may sit inside the centrifuge housing 200 which may drive the centrifuge basket 205 to spin and may collect material that is forced through the filter bag 220, the backing screen 215 and the outside wall 210. The centrifuge 200 may have a lid 235 which may be clear and a latch 240 to keep the lid 235 closed (FIG. 17).

The centrifuge 200 may be inspected to ensure it was fully cleaned prior to starting, which may also include inspecting the lid 235 as well as any corners on the centrifuge 200 as well as the drain spout 230 in the centrifuge 200. Common locations where contamination may be found are the metal fins and drain area 230 as well as the corners of the lid/sight glasses 235.

The filter bag 220 may be inserted into the centrifuge basket 205. A variety of filter bags 220 may be appropriate to be used in the system as some filter bags 220 may be more porous than other filter bags 220. The filter bag 220 may also be inspected for tears, frays, etc. and if the bag 220 is damaged, it may be replace or a manager may be alerted such as in a case of decomposition/issues with bag 220. The backing screen 215 inside the centrifuge 200 may be used to hold the filter bag 220 in place. Both the bag 220 and backing screen 215 may be inspected to ensure they are clean. Finally, the interior and exterior may be inspected for cleanliness.

The centrifuge 200 and centrifuge basket 205 may be preheated using an included heat jacket. In one embodiment, the heat jacket may be set to 50 C, heated electrically. The centrifuge basket 205 may remain at 50 C for a pre-determined time such as 30 minutes. Of course, other temperatures and times may be used and are contemplated. If the centrifuge 200 and centrifuge jacket 205 is not jacketed, the centrifuge 200 and centrifuge jacket 205 may be preheated using heat guns. Heating may be focused on outer walls of the centrifuge basket 205 to minimize high terpene that may stick to a hot inside of the centrifuge basket 205 which may hold up in the process and minimize scraping time. If heat guns are used, it may be desirable to heat to centrifuge basket 205 evenly by monitoring the temperature of the centrifuge basket walls with an IR thermometer. In some embodiments, a polypropylene backing screen 215 and filter bag 220 may be removed prior to preheating.

Referring to FIG. 1, at block 105, unactivated cannabis biomas may be extracted. The unactivated cannabis may extracted from plants. Unactivated cannabinoids are extracted via low temperature organic solvent extraction (e.g. hydrocarbon solvents such as n-butane, etc.) of fresh frozen or dry biomass. The resulting cannabinoid extract is typically rich in acidic cannabinoids and terpenes as well as some amount of natural lipids, terpenes/terpenoids, and sugars. This raw extract is purged of residual solvent At block 110, a cannabinoid solid may be precipitated in a mixture of cannabinoids, terpenes/terpenoids, and other impurities/minor components of the raw butane extract.

Budders 250 may be added to the centrifuge basket 205. A budder 250 may be a blended paste generated by thoroughly mixing the cannabinoid mixture described in 110. This budder is composed mostly of concentrated acidic cannabinoids, primarily THCa (Tetrahydrocannabinollic acid), the main chemical component in live/unactivated cannabis flower as well as a significant quantity of terpenes/terpenoids that give the final mixture a greasy consistency. Typically budders are fractionated to yield approximately 55.9% acidic cannabinoid sediment/solid, 38.5% terpenes and other dissolved compounds, and approximately 5.6% average transfer loss in fractionation but these are merely examples and other ranges are possible and are contemplated.

If separating budders 250 that are starting at room temperature, the bulk storage containers may be preheated to 60° C. in a bead bath or oven for 1 hour prior to fractionation to maximize flow rate during the centrifugation process to lower the viscosity of the terpene rich layer. Alternatively, the extract may be placed in an oven set to 70□ C for 30 minutes to 1 hour. Alternatively, rapid budder heating may be achieved using a Speedmixer warming run (1200 RPMs for 6 minutes. No vacuum). Fractionation is dependent on the amount of centrifugal force and the capillary force between the acidic cannabinoid sediment and the terpene-rich oil. The viscosity of the associated terpene rich liquid fraction of the extract is inversely related to the capillary force. By increasing temperature, the corresponding viscosity decreases and the capillary force is reduced, promoting more rapid and more efficient fractionation.

Immediately prior to loading the centrifuge basket 205, the backing screen 251 and filter bag 220 may be placed into the perforated centrifuge basket 205. The user may ensure the filter bag 220 and backing are evenly distributed and that no fabric is sticking toward the center of the basket 205.

The backing screen 215 may be fully extended but it may not overlap at all in the basket 205. The filter bag 220 may be pushed into the corners of the filter basket 220 to keep the fabric bag clear 220 of the rinsing spout. If the spout catches on the bag 220, the bag 220 may tear or shed material. The filter bag 220 may be seated securely to ensure minimal leakage wherein leakage may include material that bypasses the filter bag 229 and ensure minimal risk of the filter bag 220 catching on any part of the basket 205 or lid 235 that extends into the basket area. The filter bag 220 may be placed fully into the corners of the basket 205 to ensure even loading.

### Fractionating

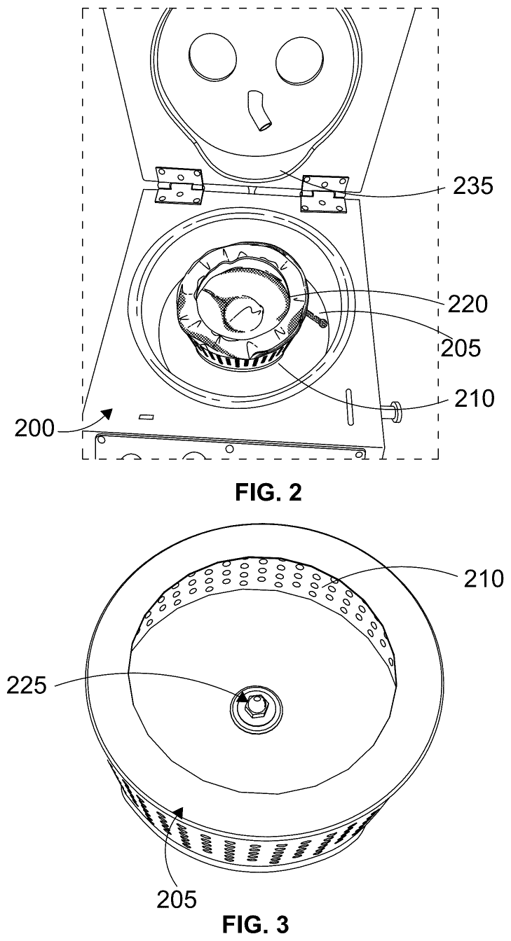

Referring to FIG. 1, at block 115, the bulk extract may be centrifugally fractionalized. The centrifugal fractionalization may include placing the extract in a batch, perforated basket centrifuge 200 such as in FIG. 2 and FIG. 8. The concentrate to be processed may be transferred to the filter bag 220. The filter bag 220 such as the bag illustrated in FIG. 5 may have a desired filter size. In one embodiment, the filter size may be 1-10 um. Logically, other filter sizes may be used depending on the desired end result, the type of input, the speed of the centrifuge, the during of the spinning, etc. Filter size most directly affects the time required to complete fractionation, as finer filters may slow filtration rate.

The filter basket 205 such as in FIGS. 5 and 16 may have a limited size. In one embodiment, the filter basket 205 may not be suitable for more than 1150 g of solid. Similarly, the a maximum load mass may be no more than 1100 g to avoid risk of damage or THCa being sprayed out of the basket into the fractionated terpene rich oil. Logically, different centrifuges 200 may have different size and weight limitations which may need to be followed.

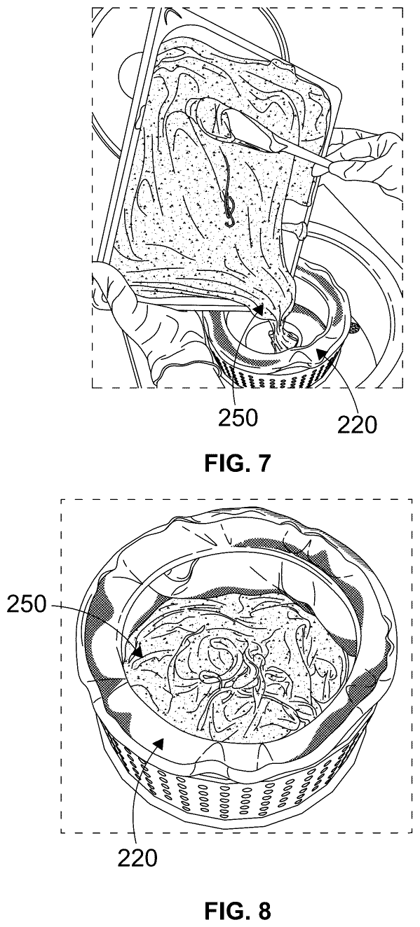

The mass transferred to the filter bag 220 such as in FIGS. 7 and 8 may be measured by weighing the initial and final weights after loading the filter bag 220. Logically, the centrifuge 200 may have a maximum load amount in terms of weight and size and there may be a desired load amount weight or size. If the filer bag 220 is overloaded, budder may be removed from the bag manually until an appropriate amount is loaded 220.

In response to a large lot of cannabis extract being fractionated, the filter bag 220 containing the cannabis biomass may be removed once the first batch of extract has been completely separated into sediment and terpene rich oil. The acidic cannabinoid sediment captured in the filter bag 220 may be removed and collected. The filter bag can then be returned to the centrifuge and filled with a second batch of the same extract lot until the entire lot has been fractionated. In this way, labor collecting the terpene rich oil from the walls of the centrifuge can be avoided until the entire lot has been fractionated.

If possible, it may be helpful to distribute material around the edges of the filter bag 220 unless extract pours similar to a liquid. This spreading may minimize risk of scraping the material with the wash pipe and contaminating the inside the system with acidic cannabinoid sediment. Additionally, uneven loading may cause vibration and lead to potential operator hazards. If vibration occurs, spread any obvious clumps of unfractionated extract as evenly as possible.

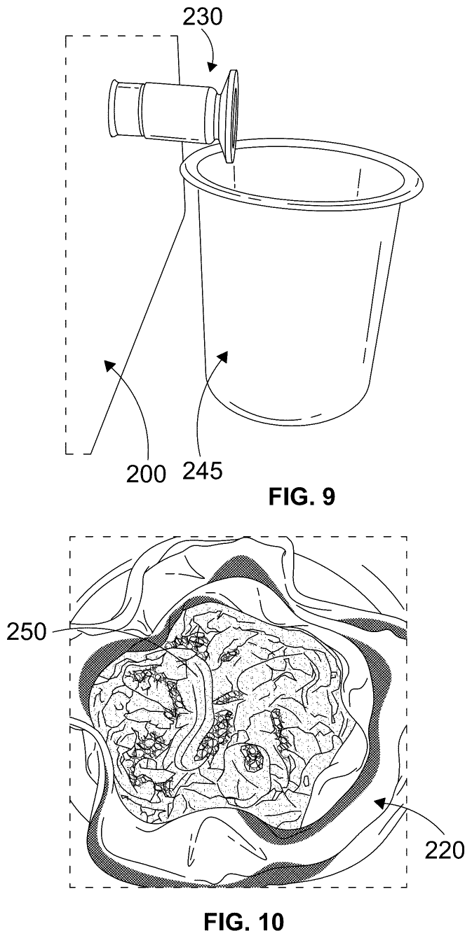

A container 245 may be cleaned to store the bulk terpene rich oil 260 which may flow out of the drain 230 of the centrifuge 200 jacket. The estimating a total volume of 35% to 45% of the weight may be estimated of the lot to be processed. The container 245 such as illustrated in FIG. 9 may be placed below the fluid outlet spout 230 on the lower or bottom side of the centrifuge 200 where gravity may force liquid to be expelled. Logically, care may be taken to ensure the container 245 does not have residue from other lots to ensure a desired quality of the final stored material. A laboratory detergent such as Alconox may be used with water if any smell remains after washing with ethanol.

Referring again to FIG. 1, at block 120, cannabinoid solids 255 may be separated from liquid terpenoid/plant fat fraction 260 and the sedimentation/centrifugation process may begin. The lid 235 of the centrifuge 200 may be closed and locked or latched using the latch 240. The centrifuge 200 may be started by pressing a start button and then promptly pressing the proper controls inputs to control the RPM of the centrifuge 200.

The RPM of the centrifuge 200 and centrifuge basket 205 may be increased slowly. The speed may be increased in steps of increases or decreased over a desired time. In one embodiment, the RPMs may start at 500 RPM for 15-20 second to allow the material in the centrifuge basket 205 to distribute along the walls. The RPMs may be slowly increased such as 100 RPM at a time over three minutes until a desired max speed is achieved which may be 4000 RPM.

The system may run for 20 minutes at 4000 RPM. In some embodiments, the centrifuge basket 205 may be slowed over a determined time period such as 5 minutes then stopping the centrifuge basket 205 rotation. The sedimentation/fractionation process may be evaluated or tested such as in FIG. 10. Testing the filter cake may include determining if the moisture of the filter cake is below a threshold, and in response to the moisture being below a threshold, stopping the centrifuge. In response to the moisture being determined to be above a threshold such as in FIG. 10, the method may be repeated. In addition, if there is significant remaining moisture in the solid cake inside the filter bag, the cake may be agitated using a silicone spatula. Care may be taken to avoid damaging the filter bag 220. If the sedimentation/fractionation process is complete, the process may proceed.

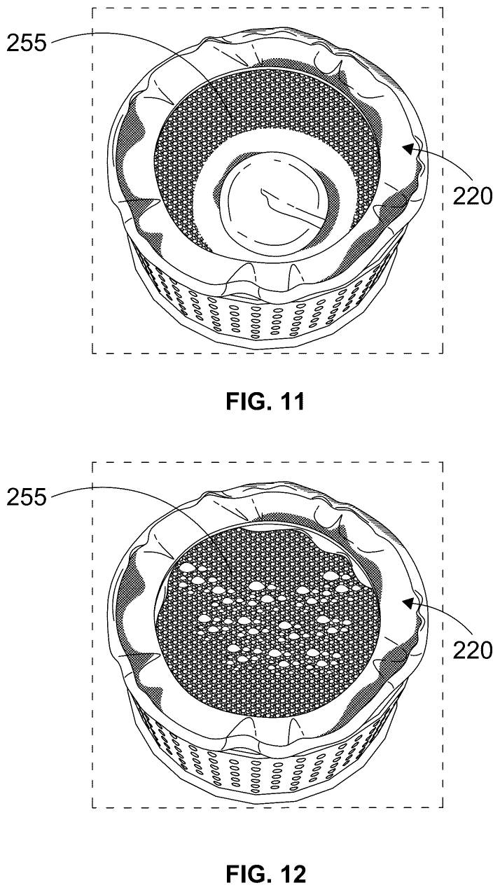

In response to the sedimentation/fractionation process being complete such as in FIGS. 11 and 12, the bag 220 may be slowly removed from the basket 220 walls which may require loosening the extract to help the bag removal process. The remaining extract may be evenly broken up with a spatula and the centrifuge process may be restarted. Care may be taken when increasing RPM of the centrifuge basket 205 as the cake may tends to distribute unevenly after agitation. If there is significant vibration/noise as RPMs are increased (following the ramp procedure in previous step), the rotation of the centrifuge 200 may be stopped, the centrifuge 200 may be opened, and the mass may be redistributed. FIG. 9/10

### Harvesting

Referring again to FIG. 1, at block 125, the extract may be reformulated to specified cannabinoid/terpenoid contents. In response to the fractionation being complete, the harvesting process may begin. If more than 1 kg is to be processed in this lot (i.e. multiple lots), the first filter bag 220 of solid acidic cannabinoids material/retentate cake material 255 may be harvested and then the bag 220 may be returned to the centrifuge basket 205 without cleaning to minimize transfer waste and to maximize throughput. An additional batch of concentrate can then be fractionated

If the HT fractionation is incomplete, such as when the surface of the cake 255 is dry but there is moist cake near the filter bag 220 as illustrated in FIG. 10, the method of FIG. 1 may be repeated after mixing the moist cake. As mentioned previously, large lots be split into 1,000 g batches and processed in sequential order 220. The process may be repeated until all solid has been processed before harvesting the high terpene extract (i.e. the filtrate) 260 such as illustrated in the progression from FIG. 10 to FIG. 11 to FIG. 12.

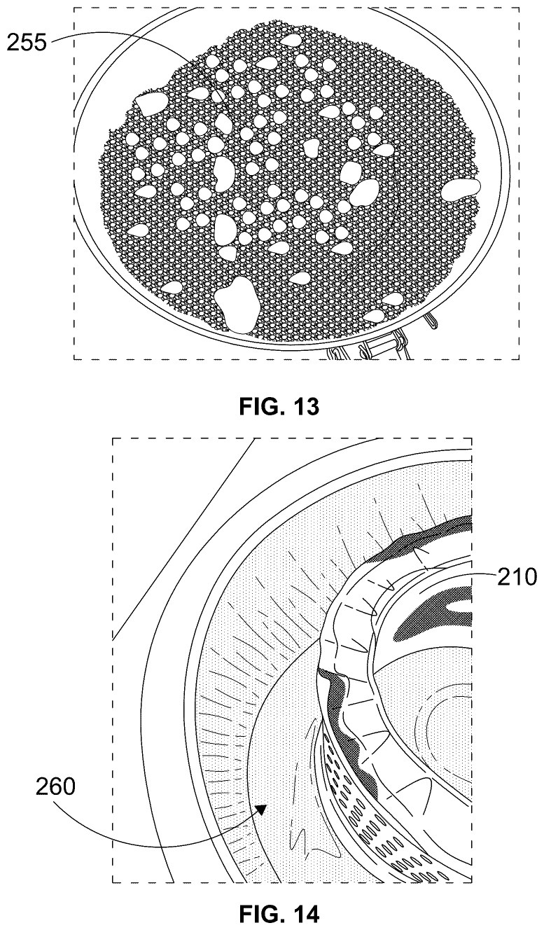

To harvest the solid concentrate (i.e. the THCa/cannabinoid isolate or cake 255) such as illustrated in FIG. 13, the filter bag 220 may be carefully removed from the basket 205. The cake 255 may be broken into small pieces as the bag 220 is pulled from the centrifuge 200. The sand/cake 255 may be moved into a final storage container, labeled with tare weight and appropriate batch identifiers. Material may be knocked from the filter bag 220 and the filter bag 220 may be returned to the centrifuge basket 205 if an additional batch of material needs to be separated.

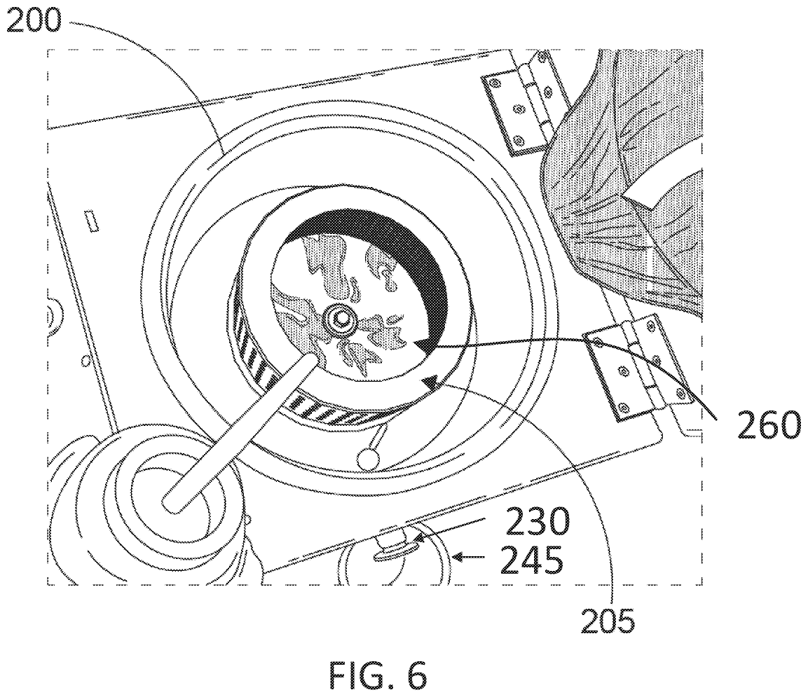

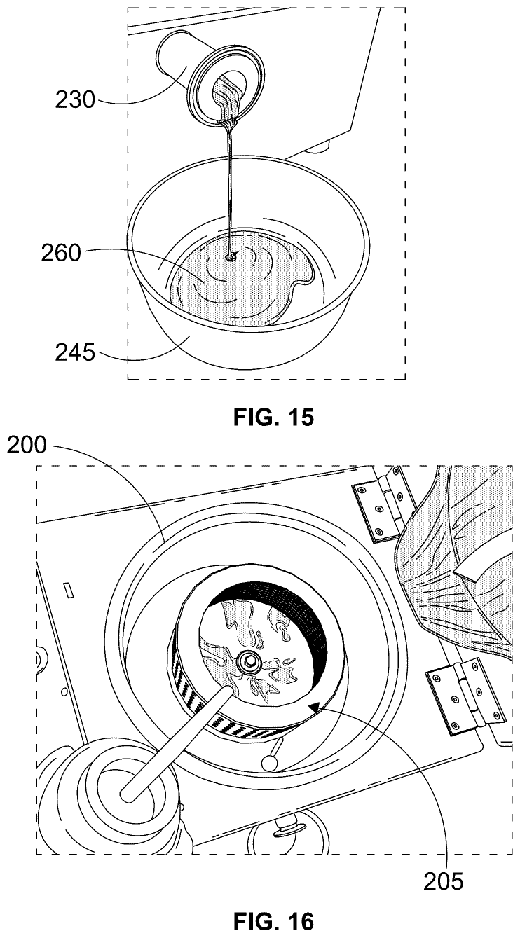

In response to all concentrate in a lot being processed, any of the high terpene fraction 260 may be harvested from the walls 210 and lid 235 of the centrifuge 200 using a stiff silicone spatula as illustrated in FIGS. 14 and 15. The majority of the high terpene extract 260 may be collected in the container 245 placed below the centrifuge outlet port 230 (FIG. 6).

The harvesting may entail moving the spatula in a circular motion around the lid 235 first, harvesting the terpene rich fraction 260 into the bottom section. The spatula may move in a circle around the upper edge of the inside of the centrifuge basket 205, moving down in a spiral, thereby moving all material to the bottom surface. The spatula may be used to scoop along the bottom surface to drain the concentrate out of the bottom outlet port 230. As much residual 260 may be removed as much as possible.

The resulting extract may include a homogenous mixture of cannabinoid solids, residual plant waxes, and terpene/terpenoids. More specifically, the resulting extract may include a cannabinoid enriched, low-terpene solid fraction 255 and a terpene enriched, low cannabinoid potency fraction 260. The a cannabinoid enriched, low-terpene solid fraction 255 may include almost entirely acidic cannabinoids. The terpene enriched, low cannabinoid potency fraction 260 may include 35-65% cannabinoids, 15-35% terpenes, and plant waxes/sugars.

To harvest the high terpene, liquid fraction 260 of the fractionated input, the basket 205 may be removed from the centrifuge 200 and the bulk high terpene fraction may be directed towards a drain port 230 such as illustrated in FIGS. 14 and 15. To ensure minimal HT 260 remains in the centrifuge 200, a small spatula may be used to harvest the last couple grams remaining in the drain tube 230. A clean and labeled container 245 may be placed under the drain port 230 to receive the fractionated high terpene extract. Typical yields for the combined fractions of the bulk terpene rich concentrate and cannabinoid isolate 260 may total approximately 95% of the input concentrate mass, depending on efficiency of recovery from both the filter bag 220 and the centrifuge walls 210. The collected material may be labeled with appropriate compliance tags.

Harvesting the basket of THCa powder 255 may include removing the filter bag 220 and emptying the cake 255 into a final container. The cake 255 may be agitated and de-lumped rapidly to ensure desirable final extract texture such as illustrated in FIG. 13. The collected material 255 may be labeled with appropriate compliance tags.

The fractions may be used to create at least one of the group including low-cost cannabinoid isolates, high terpene oils and infused products. The final product may include specific extract “textures” via direct post-separation formulation. The post-extraction fractionation/reformulation process may include pre-warming and homogenizing the extract that is being processed. In one example, the post-extraction fractionation/reformulation process further include prewarming the basket 205 centrifuge using built in jacket heater.

### Cleaning

As illustrated in FIGS. 16-18, once all product has been harvested from the system, a paper towel wet with ethanol may be used to remove any residue from the basket 205 and the area around it. The filter bag 220 and backing screen 215 may be excluded from a first wash cycle. Care may be taken to avoid having ethanol sit in the system when it is not spinning.

The filter bag 220 may be soaked in a beaker with ethanol. After soaking, the filter bag 220 may be returned to system. A spin cycle may be started with a waste container placed below the drain spout 230. The spin cycle may continue until ethanol stops pouring. The centrifuge 200 may be stopped and then wiped with paper towels and ethanol, ensure all surfaces are sprayed. This soaking process may be repeated and the residue may be wiped with a lint free cloth. The lid 235 and gasket may be sprayed down carefully to minimize cross contamination as illustrated in FIGS. 16-18.

As an example, the filter bag 220 may be placed in position with the backing screen. Ethanol, such as 100 mL may be poured over the filter bag 200 and allow to soak for 2 minutes. A spin cycle may run to 1500 rpm, and 50 mL may be added via the cake wash port, and the rotation may be stopped. The soak/spin procedure may be repeated until the bag 220 appears clean. Any remaining residue may be wiped with an ethanol soaked rag. This wash procedure may be repeated if needed. Logically, care may be taken to wipe beneath the filter basket 205 and along any corners/edges. As a final step, the inside of the outflow pipe of the drain port 230 may be wiped to ensure no residue remains in any portion of the system. The filter bag 220 may be inspected for tears/fraying that may have developed during the run.

### Troubleshooting

### Excessive Shaking/Rumbling

In response to excessive shaking/rumbling, the lid latch 240 may be checked to ensure it is secure. If the lid latch 235 is secure, the load mass may be checked to ensure the mass is approximately evenly distributed and any clumps should be broken up with a clean plastic spatula.

### High Speed Instability

In response to high speed instability, care may be taken to ensure the centrifuge 200 is level prior to operation. Significant variance from level may cause issues at high RPM.

### Shaking/Rumbling

In response to excessive shaking/rumbling, the centrifuge 200 may be stopped and causes may be investigated. In one example, the centerpost bolt 225 may be checked to verify it is tight. If the centerpost 225 bolt loosens, it may cause vibration and eventual (potentially dangerous) failure of the centrifuge 200.

### Slow Separation

In response to slow separation of the material, the basket and centrifuge walls may be heated with a heat gun (if using unjacketed system).

1. A method of fractionation of cannabis extracts via centrifugal post extraction processing comprising:
extracting unactivated cannabis biomass;
precipitating a cannabinoid solids;
centrifugally fractionalizing the bulk extract comprising:
placing the extract in a batch, perforated basket centrifuge;
separating cannabinoid solids from liquid terpenoid/plant fat fraction; and
reformulating to specified cannabinoid/terpenoid contents.

2. The method of claim 1 wherein the resulting extract comprises a homogenous mixture of cannabinoid solids, residual plant waxes, and terpene/terpenoids.

3. The method of claim 2, wherein the resulting extract comprises:
a cannabinoid enriched, low-terpene solid fraction; and
a terpene enriched, low cannabinoid potency fraction.

4. The method of claim 3, wherein the a cannabinoid enriched, low-terpene solid fraction comprises almost entirely acidic cannabinoids.

5. The method of claim 3, wherein the a terpene enriched, low cannabinoid potency fraction comprises 35-65% cannabinoids, 15-35% terpenes, and plant waxes/sugars.

6. The method of claim 5, wherein the fractions are used to create at least one of the group comprising:
low-cost acidic cannabinoid isolates,
high terpene oils,
infused products.

7. The method of claim 6, where the final product comprises specific extract “textures” via direct post-separation formulation.

8. The method of claim 1, wherein a post-extraction fractionation/reformulation process further comprises prewarming and homogenizing the extract that is being processed.

9. The method of claim 1, wherein a post-extraction fractionation/reformulation process further comprises prewarming the basket centrifuge using built in jacket heater.

10. The method of claim 9 wherein the jacket and basket are preheated to 50 C for 30 min prior to starting fractionation.

11. The method of claim 10, wherein in response to the jacket and the basket being within 5 C of target temperature prior to putting concentrate in the system, cleaning a container and placing the cleaning container at a drain port to receive fractionated high terpene extract.

12. The method of claim 1, further comprising equipping the centrifuge with a 1-10 um filter bag/strip.

13. The method of claim 12, further comprising seating the filter securely to ensure:
minimal leakage wherein leakage comprises material that bypasses the filter sheet or
minimal risk of the filter bag catching on any part of the basket or lid that extends into the basket area.

14. The method of claim 12, further comprising placing the filter fully into the corners of the basket to ensure even loading.

15. The method of claim 12, further comprising placing the extract into the basket.

16. The method of claim 15, further comprising ensuring the extract does not exceed maximum load weight (or volume) for the basket centrifuge used.

17. The method of claim 15, further comprising closing and latching the lid.

18. The method of claim 17, further comprising running the centrifuge at a predetermined speed for a predetermined time.

19. The method of claim 18, wherein the speed of the centrifuge is increased in steps over time.

20. The method of claim 18, wherein the centrifuge is stopped is vibrations over a threshold are determined.

21. The method of claim 18, further comprising slowing the centrifuge to separate the cannabis biomas for 15 minutes then stopping the centrifuge rotation.

21. The method of claim 18, further comprising after the predetermined time is over, testing the filter cake.

22. The method of claim 21, wherein testing the filter cake further comprises determining if the moisture of the filter cake is below a threshold, and in response to the moisture being below a threshold, stopping the centrifuge.

23. The method of claim 21, wherein testing the filter cake further comprises determining if the moisture of the filter cake is below a threshold, and in response to the moisture being above a threshold, repeating the method.

24. The method of claim 21, wherein in response to a large lot of cannabis extract being fractionated, the method further comprising:
removing the filter bag containing the acidic cannabinoid sediment;
harvesting the acidic cannabinoid sentiment;
placing the emptied filter bag into the centrifuge,
refilled with unfractionated extract and
repeating steps of the method until all extract has been fractionated.

25. The method of claim 1, further comprising harvesting the basket of THCa powder comprising:
removing the filter bag;
emptying the acidic cannabinoid cake into a final container.

26. The method of claim 25, further comprising agitating and de-lumping the cake rapidly to ensure desirable final extract texture.

27. The method of claim 25, further comprising harvesting the high terpene, liquid fraction of the fractionated extract by removing the basket from the centrifuge and directing the bulk high terpene fraction towards a drain port.

28. The method of claim 1, further comprising labeling the collected material with appropriate compliance tags.
